(19) United States
(12) Patent Application Publication
(10) Pub. No.: US 2006/0199262 A1
(43) Pub. Date: Sep. 7, 2006

(54) Apparatus and method for removing microbial contaminants from a flowing fluid

(76) Inventor: Dan M. Sheldon, Newberg, OR (US)

Correspondence Address: KOLISCH HARTWELL, P.C., 200 PACIFIC BUILDING, 520 SW YAMHILL STREET, PORTLAND, OR, 97204, US

(21) Appl. No.: 11/397,537

(22) Filed: Apr. 3, 2006

Related U.S. Application Data
(63) Continuation of application No. 10/407,652, filed on Apr. 4, 2003, now abandoned.
Application No. 10/407,652 is a continuation of application No. 10/216,135, filed on Aug. 8, 2002, now abandoned.
Application No. 10/216,135 is a continuation-in-part of application No. 10/032,150, filed on Dec. 20, 2001, now abandoned.
Application No. 10/032,150 is a continuation of application No. 09/479,959, filed on Jan. 10, 2000, now Pat. No. 6,333,004.

(51) Int. Cl. C12M 1/00

(52) U.S. Cl. 435/303.1; 435/809

(57) ABSTRACT

Methods and apparatuses for removing microbial contaminants from a flowing fluid in a cell culture incubator are disclosed. Some embodiments of the invention provide a cell culture incubator including a chamber, an airflow passage through which gasses circulate within the chamber, a filter configured to filter gasses that flow through the airflow passage and chamber, and a blower for circulating gasses through the airflow passage, chamber and filter. The blower includes a structural component at least partially formed from an anti-microbial material.

### CROSS-REFERENCE TO RELATED APPLICATION

This application is a continuation of U.S. patent application No. 10/407,652, filed Apr. 4, 2003 and entitled “Apparatus and Method for Removing Microbial Contaminants From a Flowing Fluid”, which is a continuation of U.S. Patent application Ser. No. 10/216,135, filed Aug. 8, 2002 and entitled “Apparatus and Method for Removing Microbial Contaminants From a Flowing Fluid”, which application is a continuation-in-part of U.S. Patent application Ser. No. 10/032,150, filed Dec. 20, 2001 which is a continuation of the U.S. patent application underlying U.S. Pat. No. 6,333,004, all of the disclosures of which are incorporated by reference herein.

### TECHNICAL FIELD

The present invention relates to an apparatus and method for removing microbial contaminants from a flowing fluid. More particularly, the invention relates to a cell culture incubator having one or more components made of an anti-microbial material.

### BACKGROUND OF THE INVENTION

The use of cell cultures is a tremendously popular research tool in a variety of scientific disciplines. The growth of cell cultures involves the in vitro growth of cells in a cell culture incubator, for example a humidified CO₂ incubator. The popularity of the technique has lead to many advances in cell growth techniques and equipment, which have made the growth of cell cultures more reliable and reproducible. However, some problems associated with cell culture growth exist despite the many recent advances made in the field. One of the most prevalent of these problems is contamination.

Many sources exist for the contamination of cell cultures. For example, any piece of equipment that a cell culture may encounter, such as an autoclave, fume hood or incubator, may introduce contaminants into the culture. Cell culture incubators are designed to provide a suitable environment for the growth of cells in culture. The primary functional components of these incubators may include any number of components, such as a chamber in which the cultures are placed for growth, a blower to circulate air in the chamber, a heating system to heat the chamber to an optimal cell growth temperature, and a filter to remove particulate contaminants from the chamber. Additionally, some incubators may include a water pan in the bottom of the chamber to humidify the cell growth environment or a CO₂ input system to control the pH of the culture. The resulting warm, moist and dark environment is perfect for the growth of cell cultures. It is also perfect for the growth of contaminants such as bacteria, mold, yeast and fungi.

Contamination can cause several types of problems in a cell culture incubator. For example, if contaminants infect a cell culture, it may ruin the culture and any experiment relying on that culture. Also, in humidified incubators, microbial contaminants in the incubator may encounter the humidity pan, and reproduce in the pan. The relative humidity inside an incubator is a function of the evaporation rate of water from the humidity pan. The rate of evaporation is dependent upon the surface area of the pan and the surface tension of the liquid in the pan. If contaminants grow in the pan, they can alter the surface tension of the water and upset the humidity characteristics of the chamber.

To prevent the contamination of a cell culture incubator, the incubator must be cleaned at regular intervals using a rigorous procedure. Even with regular cleaning, however, some locations in the incubator are particularly susceptible to contamination. One of these is the air filter. The air filter in an incubator is generally mounted on an interior surface of the chamber. The blower draws air through the filter, where the air is cleaned of particulate contaminants. Upon leaving the filter, the air flows through a conduit back into the incubator chamber, and is again cycled through the filter. One source of the contaminants removed by the filter is the opening of the chamber door by laboratory personnel. Microbial contaminants, such as bacteria and spores, enter the incubator chamber with each opening of the door. These contaminants are then drawn into the filter by the circulating air and trapped. They may then grow in the filter. Once the filter is contaminated, the potential exists for samples in the chamber to be contaminated as well.

Antibiotics may be added to cell cultures to prevent the contamination of a sample by a contaminated incubator, but they are generally not recommended for use in samples, with limited exceptions. Most antibiotics do not kill the bacteria, but only slow its growth, and thus do not remove the contaminant from the chamber. Also, the long-term use of antibiotics may alter the cultures grown in the incubator, resulting in the selective growth of antibiotic-resistant strains of cells over non-resistant strains. Furthermore, the antibiotic may be toxic to the cultured cells as well. For these reasons, it is not desirable to use an antibiotic in the cell culture to control contamination.

Some materials are known to inhibit the growth of bacteria and other microbial contaminants while showing no toxicity toward eukaryotic cells that are commonly cultured in incubators. Copper and some of its salts and oxides are among these materials. Copper compounds have long been used to control such organisms as algae, mollusks, fungi, and bacteria. Copper sulfate, for example, has many uses in agriculture. It finds its primary use in the control of fungal diseases of plants, but is also used against crop storage rots, for the control and prevention of certain animal diseases such as foot rot, and for the correction of copper deficiency in soils and animals. It also has anti-microbial uses outside of agriculture. For instance, it may be added to reservoirs to prevent the development of algae in potable water supplies. Copper sulfate, however, is not the only copper compound with antifungal and antibacterial applications. Other copper compounds, such as cuprous oxide (Cu₂O) and copper acetate (CuCH₂COOH), have also been used as fungicides. Despite its heavy use in agriculture and industry, however, neither copper nor most of its compounds commonly used in these applications have ever been shown to be toxic or to cause any occupational diseases.

Incubators have been constructed with copper chambers in the past to take advantage of the anti-microbial properties of copper compounds. However, contaminants that enter the chamber when the door is opened may still grow in areas not protected by the copper surface, such as the blower, the filter or other components. Moreover, if the filter becomes infected, the blower can spread contaminants from the filter to all other parts of the chamber. The possibility thus exists that some of these contaminants which have grown in the filter and not encountered the copper interior surface may infect cultures in the chamber.

Thus, problems exist both in inhibiting the growth of microbial contaminants in the filter of a cell culture incubator, and in segregating and retaining the inhibited contaminants away from the chamber.

### SUMMARY OF THE INVENTION

Some embodiments of the invention provide a cell culture incubator including a chamber, an airflow passage through which gasses circulate within the chamber, a filter configured to filter gasses that flow through the airflow passage and chamber; and a blower for circulating gasses through the airflow passage, chamber and filter. The blower includes a structural component at least partially formed from an anti-microbial material.

Other embodiments of the invention provide a cell culture incubator including a chamber, an airflow passage through which gasses circulate within the chamber, and a filter in fluid communication with the airflow passage, the filter having a filter element. The filter includes a first structural component at least partially constructed of a first material with anti-microbial properties, wherein the structural component is disposed within the filter upstream of the filter element so that microbial contaminants in air flowing into the incubator will contact the structural component and then be retained in the filter element. The incubator also includes a second structural component at least partially constructed of a second material with anti-microbial properties, wherein the second structural component is disposed within the airflow passage downstream of the filter element.

### BRIEF DESCRIPTION OF THE DRAWINGS FIG. 1 is an isometric view of a filter according to a first embodiment of the present invention.

FIG. 2 is a top plan view of the filter of the embodiment of FIG. 1.

FIG. 3 is a top plan view of the filter of the embodiment of FIG. 1 with the top piece removed.

FIG. 4 is an isometric view of an anti-microbial mesh according to the first embodiment of the present invention.

FIG. 5 is a sectional view taken along line 5-5 of FIG. 4.

FIG. 6 is a sectional view of an incubator showing airflow through a filter according to the present invention.

FIG. 7 is a flow diagram depicting a method of removing microbial contaminants from a flowing gas according to an embodiment of the present invention.

FIG. 8 is a flow diagram depicting a method of removing microbial contaminants from a flowing gas according to another embodiment of the present invention.

FIG. 9 is a perspective view of a blower wheel according to another embodiment of the present invention.

### DETAILED DESCRIPTION OF THE PREFERRED EMBODIMENTS

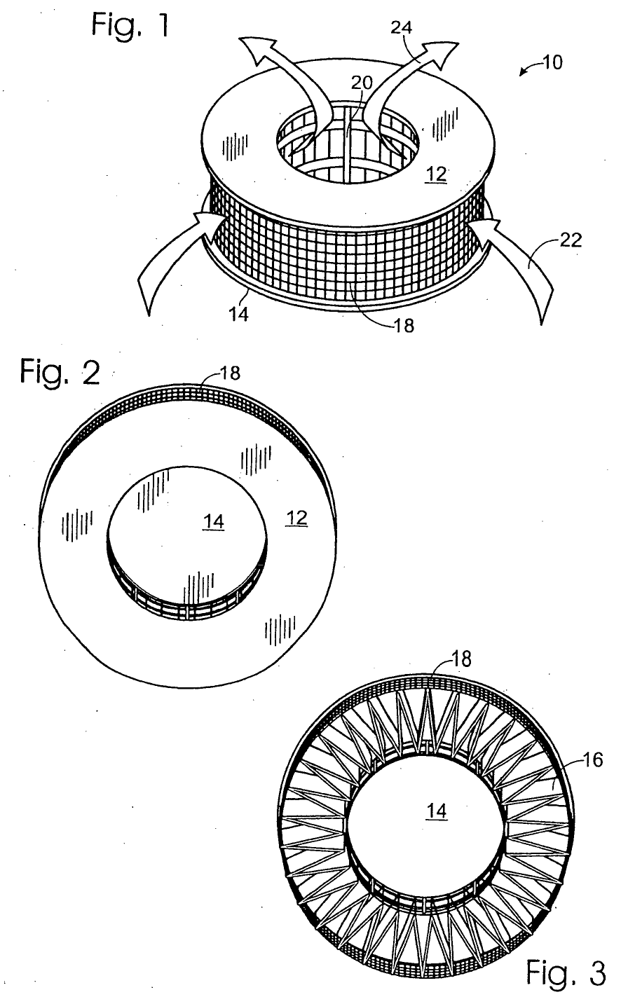

The present invention provides an apparatus and a method for removing microbial contaminants from a flowing fluid. FIG. 1 shows generally a schematic of an apparatus that may be used to practice the present invention. A filter is indicated at 10. The filter has an upper piece 12 and a lower piece 14. Upper piece 12 defines a hole in its center portion, while lower piece 14 is solid, as shown in FIG. 2, forcing air to flow out of filter 10 through the hole in upper piece 12. A filter element 16 is disposed between the upper piece and lower piece. The filter element is held in place by a mesh 18 surrounding the filter element on one side and a bracket 20 on the other side. Airflow, indicated at 22 and 24, passes through filter 10 by first passing through mesh 18, through filter element 16, and out of the hole defined by top piece 12. Top piece 12 and bottom piece 14 are joined together by mesh 18, with one edge of mesh 18 coupled to top piece 12 and the other to bottom piece 14. Top piece 12, bottom piece 14 and mesh 18 combine to form a filter casing that encloses filter element 16.

FIG. 3 shows a view of the top of filter 10 with top piece 12 removed. Filter element 16 can be seen in this view to be configured in a zig-zag pattern to maximize its surface area, and thus to maximize the speed of airflow through the filter. This may help to increase the life of the filter, as a larger surface area may clog with particulate less quickly than a smaller surface area.

To help prevent contamination, one or more structural components of filter 10 may be constructed of a material with anti-microbial properties. While many materials may be used for the structural component of the present invention, copper is a preferred material. When elemental copper metal is exposed to air, it reacts with various chemical compounds present in the air to form a variety of copper salts and oxides. For instance, in the presence of sulfur oxides, copper will form copper sulfide. In the presence of oxygen, the copper will oxidize over a period of time to Cu₂O and CuO. These compounds will generally form as a surface layer on the elemental copper metal. Additionally, water-soluble copper compounds such as copper sulfate may exist as an aqueous phase if there is any water present on the surface of the copper. Both a surface layer and an aqueous layer of the anti-microbial copper compounds will be present on any copper in the warm, moist environment of the incubator interior. The presence of these compounds on the surface of a structural component made of copper will prevent bacteria, fungi, algae, and other contaminants from growing on the element.

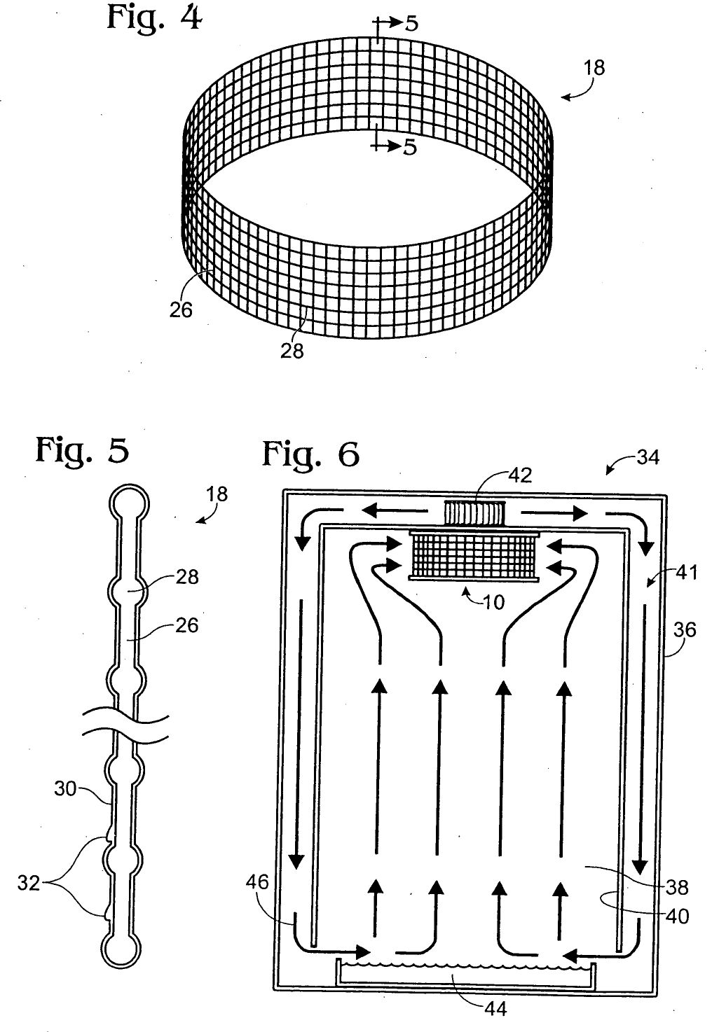

In one embodiment of the invention, a first structural component made of an anti-microbial material takes the form of mesh 18. Mesh 18 is shown separate from the rest of filter 10 in FIG. 4. Mesh 18 includes both vertical members 26 and horizontal members 28, and is configured to completely surround filter element 16. The size of the gaps defined by vertical members 26 and horizontal members 28 may be chosen to suit any particular filter or chamber design, or to accommodate particular airflow characteristics.

FIG. 5 shows a sectional view of the mesh taken along line 5-5 of FIG. 4. Though FIG. 5 demonstrates the surface condition of a mesh in a humidified incubator environment, it will be appreciated that the mesh will exhibit anti-microbial properties in any other type of incubator, including those with an extremely dry chamber environment. The view is taken as a cross-section slightly off the center of a vertical member 26, and the horizontal members 28 appear as nodes along vertical member 26. Mesh 18 typically includes a thin surface layer 30 of copper compounds covering the exposed surfaces of mesh 18. The compounds of surface layer 30 may be formed via reactions between copper and chemicals present in the air inside the incubator chamber during use, during the manufacturing process, or at any other suitable time. Among the compounds present in layer 30 will be many of the copper compounds that exhibit anti-microbial properties. Due to the moist environment inside the incubator, there also may be some moisture 32 present on the surface of mesh 18. Though droplets of moisture 32 are shown only in two places on mesh 18 in FIG. 3 for reasons of clarity, in reality moisture 32 may be found covering the entire surface, or any fraction of the surface, of mesh 18. Any water-soluble, anti-microbial copper compounds present in surface layer 30 may be found as an aqueous phase in moisture 32. In a non-humidified incubator, surface layer 30 of various copper compounds will still be present, but less moisture will be present on the surface of mesh 18.

FIG. 6 depicts the use of filter 10 in an incubator. An incubator is indicated generally at 34. Incubator 34 includes a casing 36, a chamber 38 having an interior surface 40, an airflow passage 41 defined between the casing and the chamber, a blower 42, an optional water pan 44, and filter 10. The incubator may also include a heating unit and a CO₂ source, which are not depicted in this figure. Arrows 46 indicate the direction of airflow in the incubator. Air is continuously circulated through filter 10, out blower 42, through the airflow passage 41, and back into chamber 40 at the bottom of the chamber, where it is again drawn upward toward filter 10. When the door to chamber 40 is opened to insert or remove a sample from chamber 40, contaminants present in the air, on any tools inserted into the chamber, or on the laboratory personnel using the incubator may be introduced into chamber 40. These contaminants may be drawn into filter 10 by the upward air currents created by blower 42. Upon entering filter 10, the contaminants may encounter anti-microbial mesh 18 and filter element 16. Thus, the contaminants may be trapped in filter element 16, and the copper compounds generated at mesh 18 may act to inhibit their reproduction.

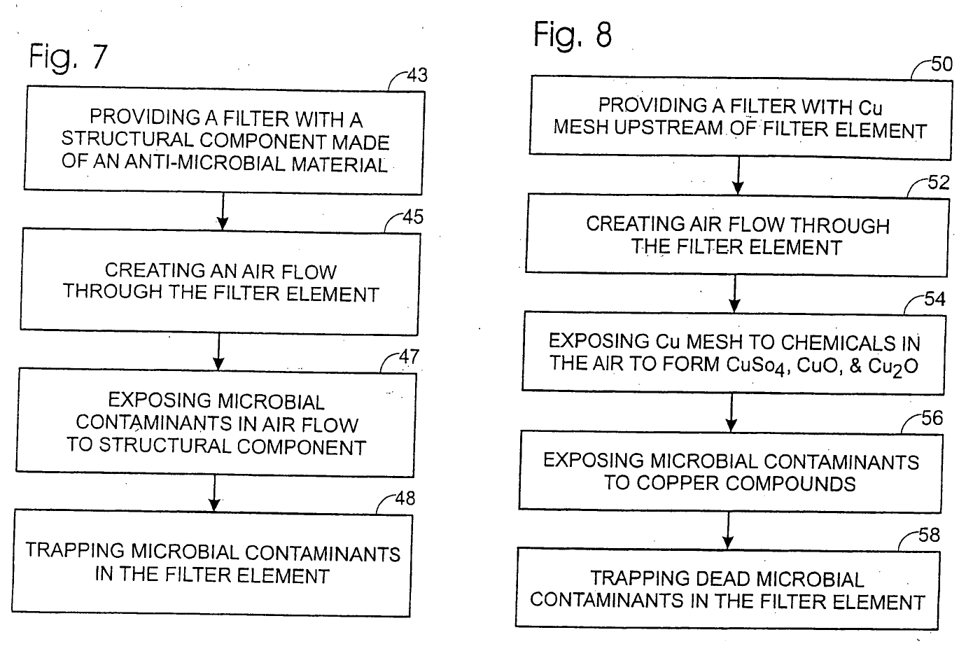

Another aspect of the present invention provides a method of removing microbial contaminants from air. The method is suited for use in any application where a sterile, microbe-free environment is desired, such as in a humidified C0₂ cell culture incubator. One embodiment of this aspect is shown in FIG. 7. First, a filter is provided at 43. According to this embodiment, the filter will have a structural component made of an anti-microbial material, and will also have a filter element. Next, a flow of air is created through the filter at 45. The flow of air may bring any microbial contaminants present in the air into contact with the anti-microbial material of the structural component, and may expose the contaminants to the anti-microbial structural component at 47. Finally, after exposing the contaminants to the anti-microbial material, the contaminants may be trapped in the filter element at 48 and thus removed from the airflow. The air downstream of the filter may thus have a lower concentration of contaminants relative to the air upstream of the filter.

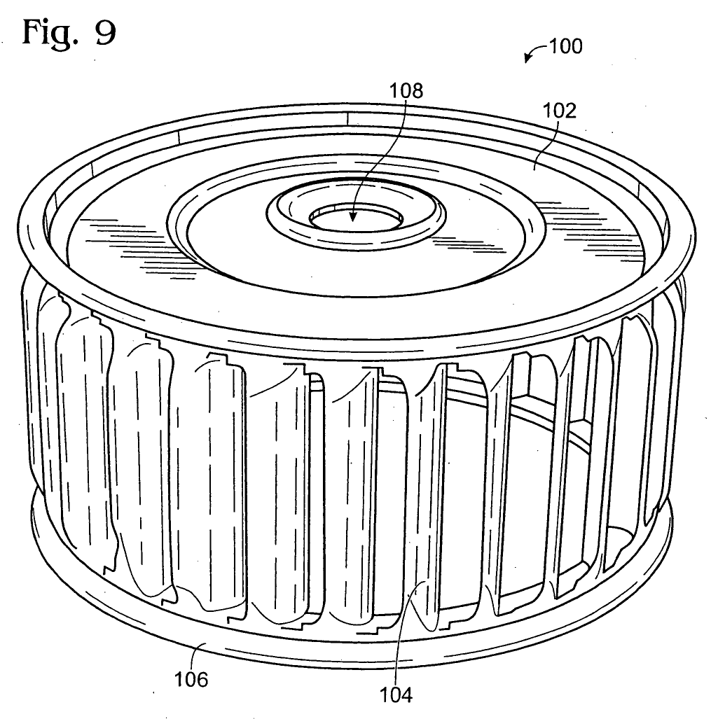

Another embodiment of this aspect of the present invention is shown in FIG. 8, which illustrates the removal of microbial contaminants from the air in a cell culture incubator. In this application, a copper mesh is provided in a cell culture incubator filter in a location upstream of the filter element at 50. Next, a flow of air is created through the filter at 52. The airflow can be created by a blower, or by any suitable pumping method. Exposure of the mesh to the air inside the incubator may result at 54 in the formation of different copper compounds, such as CuSO₄ and Cu₂O, that may display anti-microbial properties. Any microbial contaminants in the incubator may be drawn into the filter and exposed to the copper compounds at 56. Finally, the microbial contaminants may be trapped in the filter element at 58, where they may be prevented from reproducing by the presence of the copper compounds.

It is possible that some contaminants may get past mesh 18 and filter element 16 without contacting any anti-microbial compounds. These microbial contaminants may then be circulated by blower 42 through incubator casing 36 back into chamber 38, and thus may contaminate the chamber. Where chamber 38 is lined with copper, as discussed above, the microbial contaminants may not be able to find a surface within the chamber on which to reproduce. However, the contaminants may be able to find surfaces at other points between filter element 16 and chamber 38 on which to reproduce in sufficient quantities to pose a danger of contaminating cultures being grown within chamber 38. For example, surfaces on or within blower 42 may be susceptible to contamination. Because all gasses that pass through filter 10 also pass through blower 42, some contaminants that are able to get past mesh 18 and filter element 16 may find a surface within blower 42 on which to reproduce. Furthermore, blower 42 may contain some spaces that are difficult to reach for decontamination and/or cleaning.

To help prevent microbial contaminants that are able to get past mesh 18 and filter element 16 from reproducing within incubator 34, the incubator may include a second structural component made at least partially of an anti-microbial material positioned downstream of filter 10. For example, blower 42 may include one or more parts made from an anti-microbial material. Any suitable component or components of blower 42 may be made at least partially of an anti-microbial material. For example, blower 42 may utilize a bladed fan or wheel to move air within incubator 34. Because the blades of the fan or wheel contact much of the air that passes through blower 42, the surfaces of the blades may be susceptible to contamination. However, forming the blower fan or wheel at least partially from an anti-microbial material may help to prevent contaminants from reproducing on the surfaces of the wheel or fan. Furthermore, forming the blower fan or wheel at least partially of an anti-microbial material may help to kill microbial contaminants that get through mesh 18 and filter element 16 before the contaminants are circulated through incubator 34, and thus may help to prevent contamination to other parts of the incubator as well.

FIG. 9 shows, generally at 100, an exemplary blower wheel suitable for use in incubator 34. Blower wheel 100 includes a generally flat, round surface 102 from which a plurality of blades 104 extend downwardly. Blades 104 are oriented to push air from the interior of blower wheel 104 to the exterior of the blower wheel when the wheel turns. Blower wheel 100 also may include a rim 106 opposite surface 102 to which the bottom edges of blades 104 are coupled to secure the bottom edges of the blades. Furthermore, surface 102 of blower wheel 100 may include an opening 108 for attaching blower wheel 100 to the axle of a motor (not shown). Any desired part of blower wheel 100 may be formed of, coated with, or otherwise made of an anti-microbial material. For example, surfaces of blower wheel 100 that may be difficult to clean due to their close proximity to other parts of incubator 34, such as generally flat, round surface 102 and rim 106, may be coated with or formed of copper (or other suitable anti-microbial material). Likewise, the entire blower wheel 100, including surface 102, rim 106 and blades 104, may be formed from or coated with copper (or other suitable anti-microbial material) if desired. Where blower wheel 100 is only partially formed from copper, it may have any suitable construction. For example, blower wheel 100 may have a stainless steel core coated with an exterior layer of copper. The stainless steel core may be coated with copper in any suitable manner, including, but not limited to, electroplating and physical vapor deposition techniques.

Referring again to FIG. 6, blower wheel 100 may be mounted within incubator casing 36 such that rim 106 is oriented directly downstream of the outlet of filter 10 in the overall gas flow path. In this configuration, turning blower wheel 100 causes air to be drawn through filter 10, pulled through blower 42, circulated through airflow passage 41 and reintroduced into the bottom of chamber 38. Thus, substantially all the contaminants that are able to get through anti-microbial mesh 18 and filter element 16 will pass through blower wheel 100, where they may contact an anti-microbial surface of blower wheel 100, and thus may be prevented from reproducing on the surfaces of blower wheel 100. The microbial contaminants also may be killed by blower wheel 100 before being able to contaminate other surfaces within incubator 34. It will be appreciated that any other desired part of the blower besides blower wheel 100 may be made of an antimicrobial material to help inhibit contaminants from reproducing within an incubator according to the present invention. Examples of other parts of the blower that may be formed from an anti-microbial material include, but are not limited to, axles, connectors and fasteners, and casings and/or airguides that may be disposed around blower 100 to direct airflow in a desired direction. Furthermore, while the blower wheel of the depicted embodiment is positioned immediately downstream of the filter, it will be appreciated that the blower wheel may also be positioned upstream of the filter, or at any other desired location within the incubator.

While the invention has been disclosed in its preferred form, the specific embodiments thereof as disclosed and illustrated herein are not to be considered in a limiting sense as numerous variations are possible. Applicants regard the subject matter of their invention to include all novel and non-obvious combinations and subcombinations of the various elements, features, functions and/or properties disclosed herein. No single feature, function, element or property of the disclosed embodiments is essential to all embodiments. The following claims define certain combinations and subcombinations which are regarded as novel and non- obvious. Other combinations and subcombinations of features, functions, elements and/or properties may be claimed through amendment of the present claims or presentation of new claims in this or a related application. Such claims, whether they are different, broader, narrower or equal in scope to the original claims, are also regarded as included within the subject matter of applicants' invention.

What is claimed is:

1. A cell culture incubator, comprising:
a chamber;
an airflow passage through which gasses circulate within the chamber;
a filter configured to filter the gasses that flow through the airflow passage and chamber; and
a blower for circulating gasses through the airflow passage, chamber and filter, wherein the blower includes a structural component at least partially formed from an anti- microbial material.

2. The incubator of claim 1, wherein the blower is disposed within the airflow passage in such a location that substantially all of the gasses that pass through the filter also pass through the blower.

3. The incubator of claim 1, wherein the blower is disposed within the airflow passage at a location immediately downstream of the filter.

4. The incubator of claim 1, wherein the blower includes a blower wheel configured to circulate gasses through the airflow passage, and wherein the blower wheel is at least partially formed from the anti-microbial material.

5. The incubator of claim 4, wherein the blower wheel includes a steel core coated with copper.

6. The incubator of claim 1, wherein the anti-microbial material reacts with chemical compounds in the air to form products with anti-microbial properties.

7. The incubator of claim 6, wherein the anti-microbial material is copper.

8. The incubator of claim 6, wherein the products with antimicrobial properties include copper sulfate and copper oxides.

9. A cell culture incubator, comprising:
a chamber;
an airflow passage through which gasses circulate within the chamber;
a filter having a filter element, wherein the filter is in fluid communication with the airflow passage;
a first structural component at least partially constructed of a first material with anti- microbial properties, wherein the first structural component is disposed within the filter upstream of the filter element so that microbial contaminants in air flowing into the incubator will contact the first structural component and then be retained in the filter element; and
a second structural component at least partially constructed of a second material with anti-microbial properties, wherein the second structural component is disposed within the airflow passage downstream of the filter element.

10. The incubator of claim 9, wherein the incubator includes a blower, and wherein the second structural component is disposed within the blower.

11. The incubator of claim 10, wherein the second structural component is a blower wheel disposed within the blower.

12. The incubator of claim 9, wherein the first anti-microbial material is copper.

13. The incubator of claim 9, wherein the second material with anti-microbial properties is copper.

14. The incubator of claim 9, wherein the first structural component is a mesh.

15. The incubator of claim 9, wherein at least one of the first material with anti-microbial properties and the second material with anti-microbial properties reacts with chemical compounds in the air to form products with anti-microbial properties.

16. The incubator of claim 9, wherein the products with anti-microbial properties include compounds selected from the group consisting of copper sulfate and copper oxides.

17. The incubator of claim 9, wherein the first material with anti-microbial properties and the second material with anti-microbial properties are the same material.

18. The incubator of claim 9, wherein the second structural component is positioned immediately downstream of the filter.

19. A cell culture incubator, comprising:
a chamber;
an airflow passage through which gasses circulate within the chamber;
a filter configured to filter gasses circulated through the airflow passage, wherein the filter includes an inlet, an outlet, an anti-microbial structural component disposed between the inlet and the outlet, and a filter element configured to trap microbial contaminants exposed to the anti-microbial structural component; and
a blower configured to cause gasses to flow through the airflow passage, wherein the blower includes a component made at least partially from an anti-microbial material.

20. The incubator of claim 19, wherein the blower includes a bladed blower wheel at least partially formed from an anti-microbial material.
